(19) United States
(12) Patent Application Publication
(10) Pub. No.: US 2025/0064699 A1
(43) Pub. Date: Feb. 27, 2025

(54) CHLORHEXIDINE GLUCONATE-ISOPROPYL ALCOHOL WET WIPE PRODUCT AND PREPARATION METHOD THEREOF

(71) Applicant: Dukal, LLC, Ronkonkoma, NY (US)

(72) Inventor: La YANG, Dunedin, FL (US)

(21) Appl. No.: 18/725,520

(22) PCT Filed: Dec. 28, 2022
(86) PCT No.: PCT/US22/54172

§ 371 Date: Jun. 28, 2024

(30) Foreign Application Priority Data
Dec. 29, 2021 (CN) 202111647277.X

(51) Int. Cl. A61K 8/43; A61K 8/02; A61Q 17/00

(52) U.S. Cl. CPC A61K 8/43; A61K 8/0208; A61Q 17/005; A61K 2800/87

(57) ABSTRACT

A chlorhexidine gluconate-isopropyl alcohol wet wipe product includes a disinfecting solution, a nonwoven fabric and an outer pack. The disinfecting solution further contains chlorhexidine gluconate and purified water. The nonwoven fabric is configured to be impregnated with the disinfecting solution. The outer pack is configured to wrap the chlorhexidine gluconate-isopropyl alcohol wet wipe product and is a multilayer laminated film pack. The disinfecting solution also includes isopropyl alcohol. The disinfecting effect of the disinfecting wet wipe is greatly improved, the stability of the active ingredients in the disinfecting wet wipe is maintained, the effective period is prolonged, and the contents of chlorhexidine gluconate and isopropyl alcohol in the wet wipe solution in the product that act on the consumer's skin surface is consistent with the labeled amounts and achieve sufficient contents of active ingredients.

### CROSS-REFERENCE TO RELATED APPLICATIONS

The present application claims benefit of and priority to Chinese Patent Application No. 202111647277.X filed Dec. 29, 2021, the entire content of which is hereby incorporated by reference herein.

### BACKGROUND

### Field of the Disclosure

The present invention relates to the field of disinfecting wet wipes, in particular to a chlorhexidine gluconate-isopropyl alcohol wet wipe product and method for making the same.

### Related Art

Chlorhexidine gluconate, which is an efficient broad-spectrum local preservative, has strong antibacterial and bactericidal effects and wide application range. It is effective for both Gram-positive bacteria and Gram-negative bacteria, and has stronger efficacy than quaternary ammonium salt cationic surfactants. It still has a disinfecting effect in the presence of serum and blood, and has an inhibitory effect on fungi as well. It has the advantages of no irritation to tissues, good tolerance and long-lasting efficacy for human skin. After eliminating the bacteria, it can still maintain its antibacterial activity and provide protection for several hours after application. In addition, due to accumulation property, after repeated use, its disinfecting effect will enhance and its efficacy will be higher.

However, the traditional 2% chlorhexidine gluconate wet wipes on the market at present are generally prepared by directly impregnating nonwoven fabrics with a 2% chlorhexidine gluconate solution, which are then packed and sealed with an ordinary laminated film. After such products are sent to market, the volatile solution in the pack leads to unstable content of chlorhexidine gluconate, which greatly reduces the antibacterial and disinfecting effect, and also makes such disinfecting products difficult to store and short in effective period.

The wet wipes in the prior art achieve disinfection by combining nonwoven fabrics with other compounds, but there is no documentation of how to keep the active ingredients stable. The use of chlorhexidine gluconate and isopropyl alcohol as the active ingredients and the manufacturing method of the wet wipes are documented, however, there is no description of how to keep the active ingredients stable to prolong the effective period, to ensure the contents of chlorhexidine gluconate and isopropyl alcohol in the wet wipe solution that act on the consumer's skin surface during use to be consistent with the labeled amounts, and to ensure the wet wipes to have sufficient contents of active ingredients.

Accordingly, it would be beneficial to provide an antiseptic wipe that avoids these and other problems.

### SUMMARY

It is an object of the present disclosure to provide a chlorhexidine gluconate-isopropyl alcohol wet wipe product and a preparation method thereof. Isopropyl alcohol is added on the basis of a chlorhexidine gluconate disinfecting solution, and the product is packed with a novel multilayer laminated film packing material, thereby greatly improving the disinfecting effect of the disinfecting wet wipe, keeping the active ingredients of the disinfecting wet wipe stable, prolonging the effective period of the product, and ensuring the contents of the chlorhexidine gluconate and the isopropyl alcohol in the wet wipe product that act on the consumer's skin surface during use to be consistent with the labeled amounts.

In embodiments, a chlorhexidine gluconate-isopropyl alcohol wet wipe product in accordance with an embodiment of the present disclosure includes: a disinfecting solution, containing chlorhexidine gluconate and purified water; a nonwoven fabric, configured to be impregnated with the disinfecting solution; and an outer pack, configured to wrap the chlorhexidine gluconate-isopropyl alcohol wet wipe product, wherein the disinfecting solution further includes isopropyl alcohol, the outer pack is a multilayer laminated film, and in the disinfecting solution, a weight percentage of the chlorhexidine gluconate is 1.5%-3.5% and a weight percentage of the isopropyl alcohol is 2.0%-16.0%.

In embodiments, the disinfecting solution further includes a nonionic surfactant, a humectant, a pH buffer, a skin care agent and a fragrance. In the disinfecting solution, a weight percentage of the nonionic surfactant is 0.2%-1.8%, a weight percentage of the humectant is 3.0%-8.0%, a weight percentage of the pH buffer is 0.02%-0.06%, a weight percentage of the skin care agent is 0.01%-0.20%, and a weight percentage of the fragrance is 0.03-0.15%. The fragrance can heighten the pleasure of the consumer.

In embodiments, the nonionic surfactant contains polyoxyethylene (9) nonyl phenyl ether, modified polydimethylsiloxane and Tween 20, whose weight percentages in the disinfecting solution are respectively 0.1-0.9%, 0.1-0.5% and 0.02-0.1%. The nonionic surfactant has good penetrating and emulsifying abilities, and has a skin moisturizing effect.

In embodiments, the modified polydimethylsiloxane is bis-PEG/PPG-20/20 polydimethylsiloxane.

In embodiments, the humectant includes propylene glycol and glycerol, whose weight percentages in the disinfecting solution are respectively 1.5-5.0% and 1.5-3.0%.

In embodiments, the pH buffer is gluconolactone, and the skin care agent is aloe vera powder. The gluconolactone has a certain skin moisturizing effect. The aloe vera powder has a good skin moisturizing effect.

In embodiments, the nonwoven fabric includes 100% polyester fiber with a weight of 100-200 g/m².

In embodiments, the multilayer laminated film is laminated from one or more of PET, PE, OPP or aluminum foil.

In embodiments, after 24-months of storage, both the chlorhexidine gluconate content and the isopropyl alcohol content are still at least 100% of their initial values.

A method of killing 99% or more of a bacteria on a substrate comprising contacting the substrate with an amount of the chlorhexidine gluconate-isopropyl alcohol wet wipe product in an amount sufficient to kill the bacteria.

In embodiments, the bacteria are:
Staphylococcus aureus
Pseudomonas aeruginosa, or
Candida albicans.

In embodiments, a method of preparing a chlorhexidine gluconate-isopropyl alcohol wet wipe product, includes: S0: preparing or otherwise obtaining a disinfecting solution, which may include preparing the solution using the following steps: S01: adding an emulsifier-stabilizer composition into a container, and uniformly stirring the composition with a stirring apparatus until the composition becomes a uniform and thick liquid to obtain a first mixed solution; S02: adding purified water into the first mixed solution, and stirring the first mixed solution to obtain a second mixed solution; S03: sequentially adding a disinfectant-skin care agent composition into the second mixed solution, and keeping stirring to obtain a third mixed solution for later use; and S04: transferring the third mixed solution, which may be the disinfecting solution, into a storage apparatus, or other element of a production line, while stirring, and stopping stirring when the third mixed solution needs to be used; S1: putting the chlorhexidine gluconate-isopropyl alcohol disinfecting solution into a stirring tank or other container allowing stirring, and injecting the disinfecting solution through a duct or otherwise providing the solution to a container connected with or integrated into an impregnation system; S2: loading a nonwoven fabric roll onto a roller, then pulling down a nonwoven fabric, and folding the nonwoven fabric with a folding plate and a guide roller; S3: feeding the nonwoven fabric into the impregnation system containing the disinfecting solution from step S1 until the nonwoven fabric is completely soaked; S4: cutting the impregnated nonwoven fabric to form single wet wipes; S5: feeding the single wet wipes to a stacking apparatus, and pressing and stacking the single wet wipes to form a wet wipe semi-finished product; S6: sealing and storing the stacked wet wipe semi-finished product; and S7: packing the wet wipe semi-finished product with a multilayer laminated film to form an outer pack.

In embodiments, the emulsifier-stabilizer composition is a composition of polyethylene glycol nonylphenyl ether, polydimethylsiloxane and polysorbate, the disinfectant-skin care agent composition includes isopropyl alcohol, gluconic acid, propylene glycol, glycerol, gluconolactone and dried aloe vera powder, and a fragrance is added.

In embodiments, the stirring in step S01, step S02 and step S03 is stirring at a uniform speed, and the stirring is carried out at a speed of 200±20 rpm for 4±1 min.

In embodiments, in step S3, the impregnation system is provided with: a spray tube, configured to impregnate the nonwoven fabric; and a flowmeter, configured to control an amount of the solution in the duct.

In embodiments, there are 12 or 16 spray tubes.

In embodiments, in step S5, every 6 single wet wipes are pressed and stacked as a group.

In embodiments, the stacked wet wipe semi-finished products in step S6 are placed at equal intervals by an equal interval apparatus.

In embodiments, step S7 further includes sealing a side and a back of the outer pack with a heating apparatus, and an isolation zone is arranged between the stacking apparatus and a packing apparatus.

In embodiments, the multilayer laminated film is laminated from one or more of PET, PE, OPP or aluminum foil.

In embodiments, the method includes: S8: performing weight check on the packed wet wipe product by a weight detection apparatus, and performing metal detection; S9: further performing outer packaging on the packed wet wipe product; and S10: checking whether the wet wipes meet the specifications.

### BRIEF DESCRIPTION OF THE DRAWINGS

The above and related objects, features and advantages of the present disclosure will be more fully understood by reference to the following, detailed description of the preferred, albeit illustrative, embodiments of the present invention when taken in conjunction with the accompanying figures, wherein:

FIG. 1 illustrates an exemplary wipe including a nonwoven fabric configured to be impregnated with a disinfecting solution and an outer pack, before the outer pack is sealed;

FIG. 2 illustrates an exemplary flow chart showing steps for producing the exemplary wipe of FIG. 1;

FIG. 3 illustrates an exemplary flow chart showing steps for preparing a disinfection solution for use in the wipe of FIG. 1; and

FIG. 4 illustrates an exemplary production line suitable for use in implementing the steps of FIG. 2.

### DETAILED DESCRIPTION OF THE EXEMPLARY EMBODIMENTS

The specific embodiments of the present invention will be further described with reference to the following specific examples. The following examples are only intended to illustrate the technical solutions of the present invention more clearly, but not to limit the scope of protection of the present invention.

In embodiments, the wipe and method of the present disclosure provide the following advantages:
(1) A low concentration of isopropyl alcohol is added to the original chlorhexidine gluconate disinfecting solution, which effectively enhances the bactericidal effect of the chlorhexidine gluconate.
(2) The contents of chlorhexidine gluconate and isopropyl alcohol in the wet wipe solution in the product that act on the consumer's skin surface are consistent with the labeled amounts, so that the contents of chlorhexidine gluconate and isopropyl alcohol in the antibacterial wet wipe product that act on the consumer's skin surface can achieve sufficient contents of active ingredients, thereby satisfying the bactericidal requirement of the consumer's skin surface for the antibacterial wet wipe product.
(3) The outer pack of this disinfecting wet wipe product uses the multilayer laminated film, thereby improving the stability of active ingredients in the product, prolonging the effective period and facilitating storage.
(4) Meanwhile, it can be known from the experiments below that the disinfecting wet wipes prepared by using the preparation method of the disinfecting solution and the preparation method of the disinfecting wet wipes provided in the present invention have better stability and safety, and have an effective period of two or more years.
(5) In a preferred example of the present invention, the multilayer laminated film is laminated from one or more of PET, PE, OPP or aluminum foil, and is soft in texture, light-proof, airtight, non-polluting, and safe for the human body.
(6) The disinfecting solution provided in the present invention is colorless and stable, has high storage stability and good bactericidal effect, rarely causes allergic reactions, and does not react with the nonwoven fabric.
(7) The wet wipe product provided in the present invention is individually packed and convenient to use, does not need other auxiliary articles, is safe for the human body and does not produce cross-contamination.

### Product

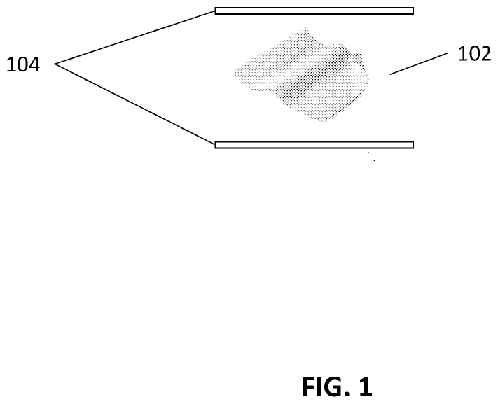

The present invention provides a chlorhexidine gluconate-isopropyl alcohol wet wipe product 100 composed of a disinfecting solution, a nonwoven fabric 102 and an outer pack 104 as illustrated in FIG. 1, for example. The disinfecting solution includes chlorhexidine gluconate and purified water. Isopropyl alcohol is added to improve the disinfecting effect of the product. As a preferred example of the present invention, in the disinfecting solution, a weight percentage of the chlorhexidine gluconate is 1.5%-3.5%, and a weight percentage of the isopropyl alcohol is 2.0%-16.0%. In embodiments, a weight percentage of the chlorhexidine gluconate is 1.5%-2.5%. In embodiments, a weight percentage of the chlorhexidine gluconate is 2.5%-3.5%. In embodiments, a weight percentage of the isopropyl alcohol is 2.0%-10.0%. In embodiments, a weight percentage of the isopropyl alcohol is 10.0%-16.0%. In one example, the disinfecting solution further includes a nonionic surfactant, a humectant, a pH buffer, a skin care agent and a fragrance. In embodiments, a weight percentage of the nonionic surfactant is 0.2%-1.8%, a weight percentage of the humectant is 3.0%-8.0%, a weight percentage of the pH buffer is 0.02%-0.06%, a weight percentage of the skin care agent is 0.01%-0.20%, and a weight percentage of the fragrance is 0.03-0.15%. In embodiments, a weight percentage of the nonionic surfactant is 0.2%-1.0%, a weight percentage of the humectant is 3.0%-5.0%, a weight percentage of the pH buffer is 0.02%-0.04%, a weight percentage of the skin care agent is 0.01%-0.10%, and a weight percentage of the fragrance is 0.03-0.10%. In embodiments, a weight percentage of the nonionic surfactant is 1.0%-1.8%, a weight percentage of the humectant is 5.0%-8.0%, a weight percentage of the pH buffer is 0.04%-0.06%, a weight percentage of the skin care agent is 0.10%-0.20%, and a weight percentage of the fragrance is 0.10-0.15%. Preferably, the nonionic surfactant contains polyoxyethylene (9) nonyl phenyl ether, modified polydimethylsiloxane and Tween 20. In embodiments, modified polydimethylsiloxane is bis-PEG/PPG-20/20 polydimethylsiloxane. In embodiments, pH buffer is gluconolactone, and the skin care agent is aloe vera powder.

In embodiments, the active ingredients with such concentrations and proportions in the product ensure the disinfecting effect with less irritation, have stable properties, and ensure the content of the chlorhexidine gluconate in the wet wipe solution that acts on the consumer's skin surface during use to be consistent with the labeled amount, so that the content of the chlorhexidine gluconate in the antibacterial wet wipe product that acts on the consumer's skin surface ensures a sufficient content of active ingredient, thereby satisfying the bactericidal requirement of the consumer's skin surface for the antibacterial wet wipe product. In embodiments, the wet wipe product 100 has the advantages of high storage stability and good bactericidal effect. In embodiments, the nonwoven fabric 102 provided in the present invention is one of the main components of the wet wipe product 100, and is impregnated with the disinfecting solution. In embodiments, the nonwoven fabric 102 used in the product is composed of polyester fiber, preferably 100% polyester fiber, however, any nonwoven fabric may be used. As a preferred solution, the nonwoven fabric 102 preferably has a weight of 100-200 g/m². After the completion of the impregnation, the product 100 may be wrapped by the outer pack 104. In order to improve the stability of the product 100 to prolong the service life, the outer pack 104 of the present invention is a multilayer laminated film laminated from one or more of PET, PE, OPP or aluminum.

### Preparation Method

In embodiments, the disinfecting solution may be prepared using steps of:

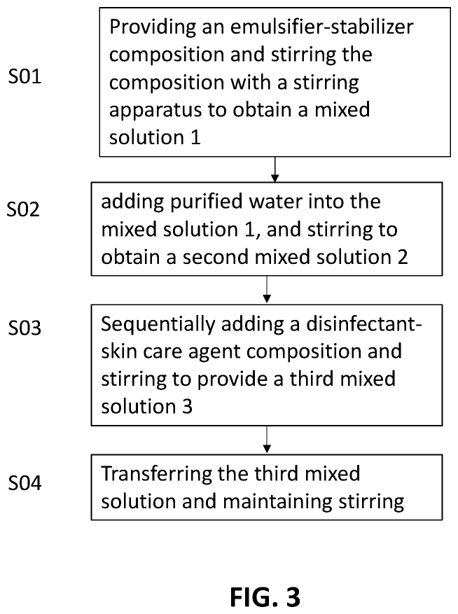

1) Providing, in step S01 (see FIG. 3), an emulsifier-stabilizer composition to a container (preferably a container capable of keeping the solute inside stable, such as a stainless steel bucket), and stirring the composition uniformly until the composition becomes a uniform and thick liquid to obtain a first mixed solution. In embodiments, the stirring may be performed with a stirring apparatus (preferably in a stirring tank). In embodiments, the emulsifier-stabilizer composition may be a composition of polyethylene glycol nonylphenyl ether, polydimethylsiloxane and POLYSORBATE-20 (of course, may also be a composition of any of the additives above), and a fragrance may be added. In embodiments, any emulsifier-stabilizer composition. In embodiments, stirring may be performed in the stirring tank at a speed of 200±20 rpm for 4±1 min.

2) Adding, in step S02, purified water to the first mixed solution 1, and stirring for 4±1 min to obtain a second mixed solution. In embodiments the stirring may take place in a liquid stirring tank or any suitable vessel.

3) Sequentially adding, in step S03, a weighed disinfectant-skin care agent composition to the second mixed solution 2, and stirring for 4±1 min to obtain a third mixed solution. Preferably, the disinfectant-skin care agent composition includes isopropyl alcohol, gluconic acid, propylene glycol, glycerol, gluconolactone and dried aloe vera powder.

4) In embodiments, in step S04, the third mixed solution may be transferred into a storage apparatus (such as a liquid storage tank) or any other suitable container, of a production line and the stirred until the third mixed solution is used. In embodiments, the third mixed solution may be the disinfecting solution. Stirring may stop when the third mixed solution is used in production.

While the disinfecting solution used in the wipe 100 is preferably provided using the above method, the method of preparing the disinfecting solution need not be a part of the method of making the wipe. In embodiments, the disinfecting solution may be premade or provided from elsewhere and used when needed to make the wipe 100. In embodiments, the disinfecting solution used in the wipe 100 may be the third mixed solution.

### Preparation of Product:

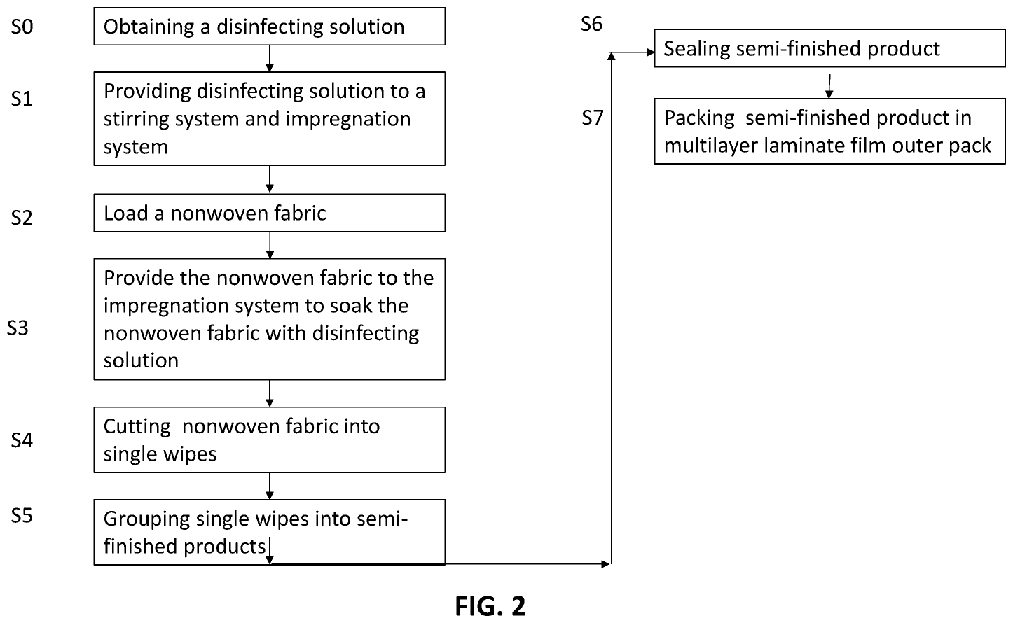

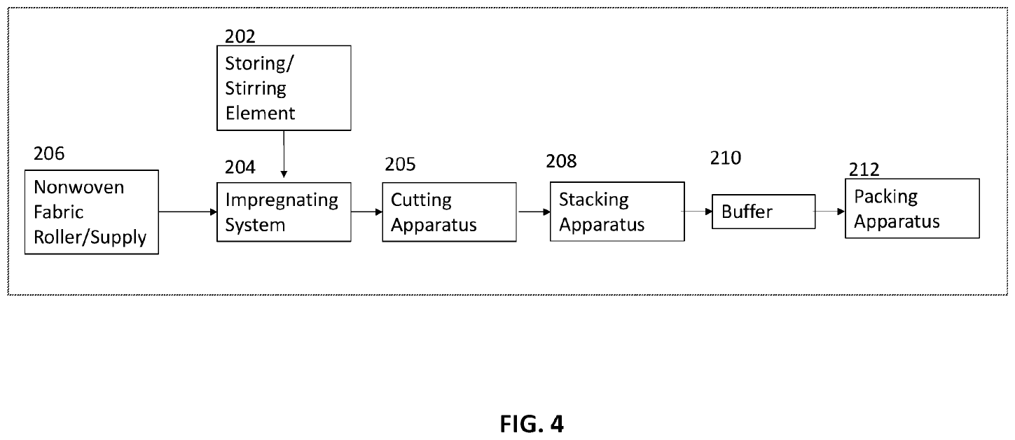

Then, in embodiments, the disinfecting solution, whether prepared by the method above or otherwise, is obtained at step S0 in FIG. 2 in a production line 200, an exemplary embodiment of which is illustrated in FIG. 4. In embodiments, the obtaining step S0 may include preparing the disinfecting solution as set forth in FIG. 3. In embodiments, the step S0 may include receiving premade disinfecting solution. In embodiments, the disinfecting solution may be put into a stirring system 202 (see FIG. 4) which may include a stirring tank such as that discussed above with respect to FIG. 3 to allow the disinfecting solution to be stirred until use in the production line 200, for example, where it is provided to an impregnation system 204 (see FIG. 4) at step S1 (see FIG. 2). In embodiments, the disinfecting solution may be injected through a duct, or otherwise provided to a liquid storage tank or other container connected with or integrated into the impregnation system 204.

In embodiments, in step S2, nonwoven fabric 202, which may be provided in a roll loaded onto a roller 206 (see FIG. 4), or other nonwoven supply element 206 in the production line, may be provided. In embodiments, the nonwoven fabric 102 may not be provided on a roller but may be provided in any suitable manner. In embodiments, in step S3, the nonwoven fabric 102 may be pulled down, and folded with a folding plate and a guide roller, or in any other suitable manner, and provided to the impregnation system 204. In embodiments, the nonwoven fabric 102 may be fed through a conveyor, or otherwise provided to the impregnation system 204, which contains the disinfecting solution and applies the disinfecting solution to the nonwoven fabric until the nonwoven fabric is completely soaked with the disinfecting solution. Preferably, the impregnation system 204 may include a spray tube (more preferably, 12 spray tubes) configured to impregnate the nonwoven fabric, and a flowmeter configured to control an amount of the solution in the duct. In embodiments, the impregnation system 204 may include more or fewer spray tubes and any suitable mechanism may be used to control the amount of solution provided. In embodiments, the impregnation system 204 may use any suitable mechanism to soak the nonwoven fabric 102 with the disinfecting solution and may not use any spray tubes.

In embodiments, the impregnated nonwoven fabric 102 may be cut to form single wet wipes at step S4, for example. The impregnated nonwoven fabric 102 may be cut with a rotary knife or other cutting element 205. In embodiments, the impregnated nonwoven fabric may not be cut at all where it is provide in a desired size and shape in which case step S4 may be omitted.

After the completion of the cutting, in embodiments, single wet wipes are fed to a stacking apparatus 208, and pressed and stacked to form a wet wipe semi-finished product in step S5, for example. Preferably, every 6 single wet wipes are pressed and stacked as a group to form a semi-finished product, however, fewer or additional single wet wipes may be pressed and stacked as a group. In embodiment, a single wet wipe may be the group and may comprise the semi-finished product.

In embodiments, the stacked group of wet wipes, that is, the semi-finished product, may be sealed and stored in step S6. Preferably, the wet wipe semi-finished product is stored in bags in a buffer zone 210 (see FIG. 4). In embodiments, the semi-finished product may not be stored in the buffer zone and step S6 may be omitted.

In embodiments, a packing apparatus 212 (see FIG. 4) may seal the stacked wet wipe semi-finished products one by one at certain intervals to ensure the ingredients are stable, so the wet wipe semi-finished products can be stored at equal intervals by an equal interval apparatus, or using the buffer zone or an isolation zone 210 arranged between the stacking apparatus 208 and the packing apparatus 212.

Finally, in embodiments, the wet wipe semi-finished product is packed with a multilayer laminated film outer pack 104 in step S7. In embodiments, the multilayer laminated film outer pack 104 is made of the multilayer laminated film packing material provided above (i.e., the multilayer laminated film laminated from one or more of PET, PE, OPP or aluminum foil). As an example, a side and a back of the multilayer laminated film outer pack 104 may be sealed with a heating apparatus, and then the multilayer laminated film outer pack 104 may be fed forward by a conveyor belt, or other device, and then cut along the sealed portions. In embodiments, the film pack 104 may hold a single wipe including the nonwoven fabric impregnated with the disinfecting solution. In embodiments, the film outer pack 104 may hold a group of wipes. In embodiments, as discussed above, the nonwoven fabric 102 sealed in the film outer pack 104 forms the product 100.

In embodiments, after cutting, each packed product 100 may be weighed using a weight detection apparatus and may be subject to metal detection to confirm that the product is the appropriate weight and contains no metal contaminants. In embodiments, outer packaging may be provided for the product 100. In embodiments, one or more products 100 may be tested to confirm that specifications are met.

### Effect and Feasibility Verification

The wet wipe product 100 prepared by the method above was tested for its efficacy by the inventors.

### 1. Stability Testing

The test was performed by chromatography. The content of chlorhexidine gluconate in the 2% chlorhexidine gluconate-isopropyl alcohol solution and the wet wipe was quantitatively detected by liquid chromatography. The content of isopropyl alcohol in the wet wipe was quantitatively detected by gas chromatography.

In this test, the contents of chlorhexidine gluconate and isopropyl alcohol were used as evaluation indexes. The contents of chlorhexidine gluconate and isopropyl alcohol were measured by a long-term experiment. Relative standard deviation (RSD) was used to determine the stability of the contents of chlorhexidine gluconate and isopropyl alcohol in the chlorhexidine gluconate-isopropyl alcohol wet wipe. The contents were respectively measured at Month 0, Month 1, Month 3, Month 6, Month 9, Month 12, Month 18 and Month 24. Based on the content at Month 0 (100%), the ratio of the content at the subsequent time points to the content at Month 0 was calculated respectively, as shown in Table 1.

As can be seen from Table 1, according to the implementation of the present invention, the contents of both chlorhexidine gluconate and isopropyl alcohol are kept stable, thereby ensuring the contents of chlorhexidine gluconate and isopropyl alcohol in the wet wipe solution that act on the consumer's skin surface during use to be consistent with the labeled amount, ensuring the contents of chlorhexidine gluconate and isopropyl alcohol that act on the consumer's skin surface to achieve sufficient contents of active ingredients, and further satisfying the bactericidal requirement of the consumer's skin surface for the antibacterial wet wipe product.

TABLE 1
Statistics on results of stability testing

| Time (Month) | Chlorhexidine gluconate | Isopropyl alcohol |
|---|---|---|
| 0 | 100% | 100% |
| 3 | 100% | 97.5% |
| 6 | 100% | 100.8% |
| 9 | 100% | 100% |
| 12 | 100% | 99.2% |
| 18 | 100% | 100.8% |
| 24 | 106.2% | 102.5% |
| RSD % | 4.91 | 6.13 |

### 2. Antibacterial Effect Testing

The wet wipe product 100 provided in the present invention was tested for its antibacterial effect on common bacteria. The results are shown in Table 2:

TABLE 2
Statistics on results of antibacterial effect testing

| | Initial | | |
|---|---|---|---|
| Type of bacteria | (CFU/ml) | Contact time 30 s | Contact time 60 s |
| Staphylococcus | 4.38 × 10⁶ | Kill 99.995% | Kill >99.9995% |
| aureus | | | |
| Pseudomonas | 2.80 × 10⁶ | Kill >99.9996% | Kill >99.9996% |
| aeruginosa | | | |
| Candida albicans | 1.92*10⁷ | Kill 99.9986% | Kill >99.9986% |

In addition, the experiments proved that on the premise of keeping the weight percentage of 2.0-3.5% chlorhexidine gluconate unchanged, the corresponding formulae with the weight percentage of the isopropyl alcohol of 6%, 8%, 12% and 16% had the same killing effect on Staphylococcus aureus, Pseudomonas aeruginosa and Candida albicans.

The above test also indicated that the sanitary wet wipe product 100 prepared by using the chlorhexidine gluconate and isopropyl alcohol satisfying “Technical Standard for Disinfection”, GB15979-2002 “Hygienic Standard for Disposable Sanitary Products” and WS575-2017 “Hygiene Requirements for Wet Wipes” had antimicrobial effect. The contents of chlorhexidine gluconate and isopropyl alcohol in the stock solution and the extruded solution of the product were consistent with the labeled amounts.

The foregoing description of the disclosed examples is provided so that those skilled in the art can implement or use this application. Various modifications to these examples will be apparent to those skilled in the art, and the general principles defined herein can be implemented in other examples without departing from the spirit or scope of this application. Therefore, this application shall not be limited to these examples shown herein, but should conform to the widest scope consistent with the principles and novel features disclosed herein. Any modification, equivalent replacement, improvement, and the like made within the spirit and principles of the present invention shall be included within the scope of protection of the present invention.

What is claimed is:

1. A chlorhexidine gluconate-isopropyl alcohol wet wipe product, comprising:
a disinfecting solution containing chlorhexidine gluconate and purified water;
a nonwoven fabric, impregnated with the disinfecting solution; and
an outer pack, configured to wrap the chlorhexidine gluconate-isopropyl alcohol wet wipe product,
wherein the disinfecting solution further comprises isopropyl alcohol, the outer pack is a multilayer laminated film, and in the disinfecting solution, a weight percentage of the chlorhexidine gluconate is 1.5%-3.5% and a weight percentage of the isopropyl alcohol is 2.0%-16.0%.

2. The chlorhexidine gluconate-isopropyl alcohol wet wipe product according to claim 1, wherein the disinfecting solution further comprises a nonionic surfactant, a humectant, a pH buffer, a skin care agent and a fragrance, and in the disinfecting solution, a weight percentage of the nonionic surfactant is 0.2%-1.8%, a weight percentage of the humectant is 3.0%-8.0%, a weight percentage of the pH buffer is 0.02%-0.06%, a weight percentage of the skin care agent is 0.01%-0.20%, and a weight percentage of the fragrance is 0.03-0.15%.

3. The chlorhexidine gluconate-isopropyl alcohol wet wipe product according to claim 2, wherein the nonionic surfactant contains polyoxyethylene (9) nonyl phenyl ether, modified polydimethylsiloxane and Tween 20, whose weight percentages in the disinfecting solution are respectively 0.1-0.9%, 0.1-0.5% and 0.02-0.1%.

4. The chlorhexidine gluconate-isopropyl alcohol wet wipe product according to claim 3, wherein the modified polydimethylsiloxane is bis-PEG/PPG-20/20 polydimethylsiloxane.

5. The chlorhexidine gluconate-isopropyl alcohol wet wipe product according to claim 2, wherein the humectant includes propylene glycol and glycerol, whose weight percentages in the disinfecting solution are respectively 1.5-5.0% and 1.5-3.0%.

6. The chlorhexidine gluconate-isopropyl alcohol wet wipe product according to claim 2, wherein the pH buffer is gluconolactone, and the skin care agent is aloe vera powder.

7. The chlorhexidine gluconate-isopropyl alcohol wet wipe product according to claim 1, wherein the nonwoven fabric is composed of 100% polyester fiber with a weight of 100-200 g/m².

8. The chlorhexidine gluconate-isopropyl alcohol wet wipe product according to claim 1, wherein the multilayer laminated film is laminated from one or more of PET, PE, OPP or aluminum foil.

9. The chlorhexidine gluconate-isopropyl alcohol wet wipe product according to claim 1, wherein after 24-months of storage the both the chlorhexidine gluconate content and the isopropyl alcohol content are still at least 100% of their initial values.

10. A method of killing 99% or more of a bacteria on a substrate comprising contacting the substrate with an amount of the chlorhexidine gluconate-isopropyl alcohol wet wipe product according to claim 1 in an amount sufficient to kill the bacteria.

11. The method of claim 10, wherein the bacteria are:
Staphylococcus aureus
Pseudomonas aeruginosa, or
Candida albicans.

12. A method of making a chlorhexidine gluconate-isopropyl alcohol wet wipe product comprises:
obtaining a chlorhexidine gluconate-isopropyl alcohol disinfecting solution;
providing the chlorhexidine gluconate-isopropyl alcohol disinfecting solution into a stirring tank, and providing the chlorhexidine gluconate-isopropyl alcohol disinfecting solution to an impregnation system;
loading a nonwoven fabric;
providing the nonwoven fabric into the impregnation system containing the chlorhexidine gluconate-isopropyl alcohol disinfecting solution until the nonwoven fabric is completely soaked;
cutting the impregnated nonwoven fabric to form single wet wipes;
feeding the single wet wipes to a stacking apparatus, and pressing and stacking the single wet wipes to form a wet wipe semi-finished product;
sealing and storing the stacked wet wipe semi-finished product; and
packing the wet wipe semi-finished product with a multilayer laminated film to form an outer pack.

13. The method of claim 12, wherein the obtaining step includes preparing the chlorhexidine gluconate-isopropyl alcohol disinfecting solution,
wherein preparing the chlorhexidine gluconate-isopropyl alcohol disinfecting solution comprises:
adding an emulsifier-stabilizer composition into a container, and uniformly stirring the composition with a stirring apparatus until the composition becomes a uniform and thick liquid to obtain a first mixed solution;
adding purified water into the first mixed solution, and stirring the first mixed solution to obtain a second mixed solution; and
sequentially adding a disinfectant-skin care agent composition into the second mixed solution, and maintaining stirring to obtain a third mixed solution.

14. The method of claim 13, wherein the preparing step includes transferring the third mixed solution to the stirring tank and stirring the mixed solution until it is used.

15. The method of claim 12, wherein the emulsifier-stabilizer composition is a composition of polyethylene glycol nonylphenyl ether, polydimethylsiloxane and polysorbate, and
the disinfectant-skin care agent composition includes isopropyl alcohol, gluconic acid, propylene glycol, glycerol, gluconolactone and dried aloe vera powder, and a fragrance is added.

16. The method of claim 13, wherein stirring is carried out at a speed of 200±20 rpm for 4±1 min.

17. The method of claim 12, wherein the impregnation system comprises:
a spray tube, configured to impregnate the nonwoven fabric; and
a flowmeter, configured to control an amount of the chlorhexidine gluconate-isopropyl alcohol disinfecting solution soaking the nonwoven fabric.

18. The method of claim 17, wherein the impregnation system comprises 12 or 16 spray tubes.

19. The method of claim 12, wherein the packing step includes sealing a side and a back of the outer pack with a heating apparatus.

20. The method of claim 12, wherein the multilayer laminated film is laminated from one or more of PET, PE, OPP or aluminum foil.
